(19) United States
(12) Patent Application Publication
(10) Pub. No.: US 2007/0265246 A1
(43) Pub. Date: Nov. 15, 2007

(54) Treatment of an Intestinal Adenoma and/or Adenocarcinoma by Inhibition of Notch Pathway Activation

(76) Inventors: Johannes C. Clevers, Huis Ter Heide (NL); Maria Elisabeth Van Gijn, Maarssen (NL); Johannes Hendrikus Van Es, Maarssen (NL)

Correspondence Address: TRASK BRITT, P.O. BOX 2550, SALT LAKE CITY, UT, 84110, US

(21) Appl. No.: 11/667,419

(22) PCT Filed: Nov. 10, 2005
(86) PCT No.: PCT/NL05/00788

§ 371 Date: Jul. 26, 2007

(30) Foreign Application Priority Data
Nov. 10, 2004 (EP) 04078090.0
Apr. 6, 2005 (EP) 05075806.9

(51) Int. Cl. A61K 31/55; C12N 5/06

(52) U.S. Cl. 514/217.07; 435/375

(57) ABSTRACT

The invention relates to the field of biochemistry and medicine. More specifically the invention relates to methods and pharmaceuticals for the treatment of intestinal neoplasia. The invention discloses the surprising finding that inhibitors of Notch pathway activation (for example γ-secretase inhibitors) are extremely useful in the treatment of intestinal adenoma and/or adenocarcinoma.

The invention relates to the field of biochemistry and medicine. More specifically the invention relates to methods and pharmaceuticals for the treatment of an intestinal adenoma (also termed a polyp) and/or adenocarcinoma.

The small intestine is the largest component of the digestive tract and the major site of digestion and absorption. In addition to receiving chyme from the stomach, the initial segment of the small intestine, the duodenum, receives bile from the gall bladder and digestive enzymes from the pancreas. The pancreatic enzymes are produced in an inactive form and only become active in the lumen of the duodenum. The small intestine is divided into three parts, the duodenum, the jejunum and the ileum.

The luminal surface is completely covered by a number of finger- or leaf-like projections called villi, 0.5-1.5 mm in length. The core of a villus is an extension of the lamina propria, and its surface is covered by a simple columnar epithelium. Opening onto the luminal surface at the bases of the villi are simple tubular structures called intestinal glands or crypts of Lieberkuhn. The crypts extend downward toward the muscularis mucosae. The simple columnar epithelium lining them is continuous with that covering the villi. The predominant cell type of the epithelium is the enterocyte or absorptive cell. Each enterocyte has about 3000 microvilli at its luminal surface, which appear in the light microscope as the fuzzy striated border on the surface of the villi. The villi and microvilli, together with folds in the submucosa called plicae circulares, increase the absorptive surface of the small intestine about 600 times.

The epithelium of the small intestine consists of the following cell types: enterocyten, goblet cells, Paneth cells, enteroendocrine cells, microfold cells and undifferentiated cells. Some of these cells will be discussed in more detail.

Enterocytes (also called absorptive cells) are tall columnar cells with microvilli and a basal nucleus, specialized for the transport of substances. They are bound to one another and other cell types by junctional complexes. Amino acids and monosaccharides are absorbed by active transport, monoglycerides and fatty acids cross the microvilli membranes passively. Absorbed substances enter either the fenestrated capillaries in the lamina propria just below the epithelium, or the lymphatic lacteal (most lipids and lipoprotein particles). Enterocytes have a lifespan of about 5-6 days.

Goblet cells are mucus-secreting cells and are the second most abundant epithelial cell. They are found interspersed among the other cell types. Their mucus is a very large glycoprotein that accumulates at the apical end of the cell. The slender base of the cell holds the nucleus and organelles. The abundance of goblet cells increases from the duodenum to the terminal ileum. Their lifespan is also 5-6 days.

Undifferentiated cells are stem cells and are found only at the base of the crypts and give rise to all the other cell types. A cell destined to be a goblet cell or enterocyte undergoes about 2 additional divisions after leaving the pool of stem cells, and migrates from the crypt to the villus. It will be shed at the tip of the villus.

The large intestine consists of the colon, cecum, appendix, rectum and anal canal. The principal functions of the colon are the reabsorption of electrolytes and water and the elimination of undigested food and waste. The mucosa appears smooth at the gross level because it has no villi. Numerous straight, tubular glands are present. They extend all the way to the muscularis mucosae. The glands and the surface are lined with simple columnar epithelium whose cell types are as described for the small intestine. However Paneth cells are usually absent in the adult human and enteroendocrine cells are rare. Columnar absorptive cells and goblet cells are abundant. Goblet cells are more prevalent in the crypts than along the surface, and their number increases distally toward the rectum. The mucus facilitates the passage of the increasingly solid colonic contents, and covers bacteria and particulate matter. The absorptive cells have short, irregular microvilli, and although they secrete a glycocalyx, it has not been shown to contain digestive enzymes. The absorptive cells actively transport electrolytes. Water is also absorbed as it passively follows the electrolytes. As in the small intestine, undifferentiated cells are found at the base of the crypts.

Despite extensive knowledge relating to the multitude of cancer forms (varying in appearance from solid tumours and related metastases in distinct parts of the body to leukaemia's of blood cells that circulate throughout the body, and varying from being totally benign to being aggressively malignant) effective therapy of cancer is difficult and in general restricted to three types; treatment via radiation, via chemotherapy and via surgery.

Possibilities for a more specific therapy, directed against the underlying cause of a specific cancer or group of cancers are currently virtually non-existing. Extensive efforts are directed at providing such specific drugs through drug discovery attempts that try to identify candidate drugs for specific cancer therapy.

Development of cancer often starts with changes in a cell that lead to the unrestricted development and division of that first cell into an ever dividing population of cells. These changes are often an accumulation of mutations or other alterations in key genes that occur chronologically, whereby the mutated cell population looses its original, often specialised character and acquires more and more of a cancerous nature. Normal processes of growth regulation of cells are dysfunctioning in the altered cells. Transcription of genes that are normally only little expressed in said cell type is in cancerous cells no longer controlled.

Activation of transcription of genes by transcription factors that would otherwise be dormant in the specific cell type can for example lead to the so typical unrestricted growth and neoplastic nature of cancer.

Examples are mutations in suppresser genes that function normally by generating proteins that are suppressing transcriptional pathways which are no longer of use in a specialised cell. Mutated suppresser genes do no longer help keep the growth of a cell at bay. Drugs directed against or intervening with the specific protein-protein or protein-DNA interactions in transcriptional pathways controlling cell growth or development can be considered typical candidate drugs for use in specific cancer therapy, especially when such pathways have gone awry and lead to unrestricted growth cells.

A typical example of a transcriptional pathway gone wrong and leading to development of cancer, can be found with adenomatous polyposis coli (APC). Mutations in this gene are among the most common disease-causing events in humans, approximately 50% of the population will, during a normal life span, develop colorectal polyps initiated by APC mutations. Individuals who inherit APC mutations develop thousands of colorectal tumours. APC protein interacts with at least six other proteins; β-catenin, γ-catenin, tubulin, EB1, hDLG and ZW3/GSKβ kinase that may be involved with communicating APC related growth control. Colon carcinoma cells with mutant APC contain large amounts of monomeric, cytoplasmic β-catenin. Reintroduction of wild-type APC reduces the overall amount of β-catenin.

Especially during the last decade the molecular genetic analysis of colorectal cancer has revealed that the adenomatous polyposis coli (APC) tumor suppresser gene, originally identified as the gene responsible for familial adenomatous polyposis (FAP), plays a rate-limiting role in colorectal tumour formation: it is mutated in the majority of sporadic colorectal tumours and inactivation of both APC alleles occurs at early stages of tumour development in mouse and man.

Moreover, although the colorectal tumours represent the hallmark of FAP, germline APC mutations often result in a broad spectrum of lesions of ecto-, meso- and endodermal origin. In fact, FAP patients are at high risk for the development of desmoids (fibromas), duodenal and gastric tumors, congenital hypertrophies of the retinal epithelium (CHRPEs), epidermal cysts, osteomas, CNS tumours, and others. The latter observation clearly indicates that the APC gene plays a critical role in the maintenance of tissue homeostasis at many different sites throughout the organism.

Notch signalling controls spatial patterning and cell fate decisions throughout the animal kingdom (Artavanis-Tsakonas et al 1999). The Notch genes encode large, single transmembrane receptors. Interaction between Notch receptors and ligands results in proteolytic cleavages of the receptor. The resulting free Notch intracellular domain (NICD) translocates into the nucleus, where it binds to the transcription factor RBP-Jκ (CSL or CBF1), thus activating target gene transcription (Baron 2003, Mumm & Kopan 2000). The best-characterized Notch target genes are the hairy/enhancer of split (HES) transcriptional repressors. The HES proteins in turn repress expression of downstream genes (Heitzler et al 1996, Oellers et al 1994).

While Notch pathway components are expressed in mouse intestine (Schroder & Gossler 2002), genetic evidence for the involvement of these components in the control of epithelial cell fates is currently not available. Animals deficient in HES-1, known to represent a Notch target gene in other tissues, show a relative increase in mucosecreting and enteroendocrine cells at the expense of adsorptive cells (Jensen et al 2000). A putative downstream target of HES-1 repression in the intestine, Math-1 (Jensen et al 2000) (Zheng et al 2000) is required for commitment towards the secretory linage as revealed by gene knockout (Yang et al 2001). These results have been interpreted to indicate that Notch signalling skews the fate of differentiating crypt cells leaving the transit amplifying compartment towards an enterocyte phenotype. In this scheme, Notch signalling activates transcription factor genes such as HES1, which in turn repress genes like Math1, driving differentiating cells away from the secretory lineage.

Indirect support for the control of intestinal cell fate by Notch stems from the use of gamma-secretase inhibitors, as originally developed for Alzheimer's disease. Notch is one of several known γ-secretase substrates. Proteolytic processing of Notch by γ-secretase is an essential step following activation of the pathway. As a consequence, one of the effects of γ-secretase inhibitors is the abrogation of Notch pathway activation (De Strooper et al 1999, Kopan & Goate 2000). Rodent toxicological studies with these inhibitors have revealed increases in size and number of mucosecreting goblet cells (Searfoss et al 2003; Wong et al 2004; Milano et al, 2004). Due to these kinds of studies multiple promising γ-secretase inhibitors have been stopped in their further clinical development for the treatment of Alzheimer, because they are heavily suspected of inducing intestinal abnormalities.

The invention discloses the surprising finding that inhibitors of Notch pathway activation (for example γ-secretase inhibitors) are extremely useful in the treatment of an intestinal adenoma and/or adenocarcinoma. Treatment of an intestinal adenoma and/or adenocarcinoma with an inhibitor of Notch pathway activation results in the inhibition of proliferation of the transformed/malignant cells and results in their differentiation into postmitotic (i.e. no longer (visibly) dividing) cells such as goblet cells. These differentiated cells have a relative short lifespan (5-6 days) and the body clears them after their death and the intestinal adenoma and/or adenocarcinoma is/are at least in part decreased in size/volume.

In a first embodiment the invention provides a method for modifying the fate of an adenoma and/or adenocarcinoma cell comprising influencing Notch pathway activation. More specifically, the invention provides a (in vitro and/or an in vivo) method for inducing the formation of a postmitotic cell from an adenoma and/or adenocarcinoma cell comprising at least in part inhibiting Notch pathway activation in said adenoma and/or adenocarcinoma cell. An example of a postmitotic cell is a goblet cell.

In a further embodiment the invention provides a method for at least in part decreasing intestinal adenoma and/or adenocarcinoma present in an animal comprising at least in part inhibiting Notch pathway activation in said animal.

Notch pathway activation typically goes along the following events. Notch is a transmembrane surface receptor that can be activated through multiple proteolytic cleavages, one of them being cleavage by a complex of proteins with protease activity, termed γ-secretase. Gamma (γ)-secretase is a protease that performs its cleavage activity within the membrane. Gamma (γ)-secretase is a multicomponent enzyme and is composed of at least four different proteins, namely, presenilins (presenilin 1 or 2), nicastrin, PEN-2 and APH-1. Presenilin is the catalytic centre of γ-secretase. On ligand binding the Notch receptor undergoes a conformational change that allows ectodomain shedding through the action of an ADAM protease which is a metalloprotease. This is followed immediately by the action of the γ-secretase complex which results in the release of the Notch intracellular domain (NICD). NICD translocates to the nucleus where it interacts with CSL (C-promoter-binding factor/recombinant signal-sequence binding protein Jκ/Supressor-of-Hairless/Lag1). The binding of NICD converts CSL from a transcriptional repressor to an activator which results in the expression of Notch target genes.

The present inventors have studied the expression of various Notch pathway components and target genes in adenomas that spontaneously occur in the APC-mutant min mouse, a reliable animal model for familial adenomatous polyposis and for intestinal cancer. The inventors established expression in adenomas of multiple components of the Notch pathway, including Notch2 and Delta-like-1. Moreover, the inventors determined that the Notch target gene Hes1 is expressed in adenomas, which indicates that active Notch signalling occurs in these adenomas. In a next step the inventors have determined the effect of inhibitors of Notch pathway activation on adenomas.

By providing an inhibitor of Notch pathway activation to a malignant/transformed cell involved in intestinal adenoma and/or adenocarcinoma, the fate of said cell is changed towards a postmitotic fate, e.g. to a goblet cell type. Such a cell has a relative short lifespan (5-6 days) and will die shortly after providing the inhibitor, resulting in a decreased amount of intestinal adenoma and/or adenocarcinoma cells, i.e. a decreased volume of least one adenoma and/or adenocarcinoma.

By providing an inhibitor of Notch pathway activation the proliferative capacity of intestinal adenoma and/or adenocarcinoma is at least in part decreased. Preferably, the decrease is such that it is visible with a scan or an exploratory operation. Even more preferably, the decrease of the intestinal adenoma and/or adenocarcinoma is complete, i.e. no (visible) remaining malignant/transformed cells.

An animal is herein defined as a non-human animal or a human being.

Inhibition (of at least part, but preferably completely) of Notch pathway activation is accomplished in different ways, which are (non-limiting) outlined here under. Preferably the inhibition is performed locally, i.e. in an adenoma and/or adenocarcinoma cell, without interfering with Notch pathway signalling in a non-adenoma and/or non-adenocarcinoma cells.

Moreover, Notch pathway activation is defined to include pathway activation of Notch-like molecules and/or different allelic variants of Notch (-like) molecules.

An intestinal adenoma is typically defined as a benign tumor such as a polyp. An adenocarcinoma is typically defined as a malignant tumor and is also referred to as colorectal cancer. The term “intestinal adenoma and/or adenocarcinoma” is herein defined to also comprise metastasis derived from said adenocarcinoma. Preferably, said metastasis is derived from an intestinal adenocarcinoma. Such a metastasis may be located anywhere in the body of the (to be) treated subject, for example an intestinal adenocarcinoma metastasis located in the lung or brain or kidney or liver of said subject.

Moreover, a method of the invention is suitable in the treatment an intestinal adenoma and/or adenocarcinoma and/or a metastasis derived thereof independent of the size. One disadvantage of currently used tumor therapy is that said therapy often needs to rely on the presence of newly formed blood vessels (angiogenesis) to deliver the compound used in said therapy to said tumor or a metastasis thereof. The present invention relies on the inhibition of proliferation of transformed/malignant cells and their differentiation into post-mitotic cells. Hence, a small tumor or a small metastasis (without yet any new blood vessels formed) can be treated at a much earlier time point compared to some of the traditional therapies.

In a preferred embodiment the invention provides a method for at least in part decreasing an intestinal adenoma and/or adenocarcinoma present in an animal comprising at least in part inhibiting Notch pathway activation in said animal wherein said Notch pathway activation is at least in part inhibited by providing said animal with a γ-secretase inhibitor. Such a γ-secretase inhibitor is for example peptidic in nature or non-peptidic or semi-peptidic and is preferably a small molecule. Gamma-secretase inhibitors were originally defined for Alzheimer's disease. A key step in the pathogenesis of Alzheimer's disease is APP proteolysis resulting in the formation of the amyloid-β peptide (Aβ), the principle protein component of the characteristic cerebral plaques of the disease. APP ((just like Notch) is first cleaved in the extracellular domain (in this case by a β-secretase) and the remaining part of APP is cleaved within the membrane by γ-secretase to produce Aβ peptide. Inhibition of Aβ peptide production by blocking γ-secretase activity is at present one of the most promising therapeutic strategies to slow progression of Alzheimer's disease pathology. Several companies have in the meantime developed γ-secretase inhibitors, such as DAPT (N-[N-(3,5-difluorophenylacetyl)-L-alanyl]-S-phenylglycine t-butyl ester). Also compounds from the chemical classes AS (arylsulfonamide), DBZ (dibenzazepine (DBZ), BZ (benzodiazepine), LY-411,575 and many others, have been tested for their γ-secretase inhibiting activity. An overview in respect of γ-secretase inhibitors is for example outlined in Harrison et al 2004 in which the γ-secretase inhibitors have been divided in solfonamides/sulfones and benzodiazepines/benzolactams. Several of these γ-secretase inhibitors have already been in clinical phase I and II trials. It is clear to a skilled person that a method for at least in part decreasing an intestinal adenoma and/or adenocarcinoma present in an animal comprising at least in part inhibiting Notch pathway activation in said animal wherein said Notch pathway activation is at least in part inhibited by providing said animal with an inhibitor of γ-secretase, can be performed by providing at least one or at least two or more γ-secretase inhibitors, i.e. by providing a combination of different γ-secretase inhibitors. It is clear that γ-secretases are typically capable of acting in (at least) two pathways: the APP and the Notch pathway. Chemical companies have in the meantime developed γ-secretases which are specific for one or the other, e.g. γ-secretases that are specific for the APP pathway and do not interfere with the Notch pathway. It is clear that γ-secretases that do not interfere with the Notch pathway are not useful in a method of the invention. Hence, preferably a γ-secretase capable of interfering in the Notch pathway as well as in the APP pathway or γ-secretases capable of specifically interfering in the Notch pathway are preferred. Such inhibitors can for example be found in Harrison et al 2004, which are incorporated herein by reference.

In a preferred embodiment the invention provides a method for at least in part decreasing an intestinal adenoma and/or adenocarcinoma present in an animal comprising at least in part inhibiting Notch pathway activation in said animal wherein said Notch pathway activation is at least in part inhibited by providing said animal with a γ-secretase inhibitor, wherein said inhibitor of γ-secretase is DAPT or dibenzazepine (DBZ) or benzodiazepine (BZ).

DAPT, DBZ or BZ are both effective in inducing the formation of a postmitotic cell from an adenoma and/or adenocarcinoma cell and these compounds thus have a similar effect in a method according to the invention. DBZ however has an IC50 of 1.7 nM, BZ has an IC50 of 2.2 nM, while DAPT has an IC50 of 10 nM, and hence lower amounts of DBZ or BZ can be used if compared to DAPT for obtaining similar results.

As already outlined above γ-secretase is a complex of proteins. Another way of at least in part inhibiting Notch pathway activation is accomplished by at least in part inhibiting the formation of said complex of proteins because only the complex is considered to be active. This is for example accomplished by providing one of the components as a dominant negative molecule or by providing a part/molecule of said complex which part/molecule comprises a mutation preventing further complex formation or resulting in an unstable (non-active) protein complex. Yet another way of at least in part inhibiting Notch pathway activation is by specifically inhibiting the catalytic part of said complex, i.e. specific inhibition of the presenilins.

In another preferred embodiment the invention provides a method for at least in part decreasing an intestinal adenoma and/or adenocarcinoma present in an animal comprising at least in part inhibiting Notch pathway activation in said animal, wherein said Notch pathway activation is at least in part inhibited by at least in part diminishing ligand mediated activation of Notch. As outlined above, Notch pathway activation starts with ligand binding after which event the Notch receptor undergoes a conformational change that allows ectodomain shedding through the action of an ADAM protease. It is clear to a skilled person that the ligand binding to Notch may be at least partly, but preferably completely, inhibited by multiple strategies. Preferably said ligand-mediated activation of Notch is at least in part diminished by providing said animal with a dominant-negative ligand of Notch. Examples of natural Notch ligands are the proteins Delta, Jagged and Serrate. Dominant negative ligands, i.e. ligands capable of binding to Notch essentially without further activation of Notch pathway (blocking of Notch pathway activation), may be derived from said natural ligands, for example by producing small binding molecules based upon the binding part of said natural ligand. In case such dominant negative ligands are brought into contact with Notch, binding of said dominant negative ligand to Notch takes place without further activation of the Notch pathway. Preferably, said dominant negative ligand sticks/binds for longer periods of time to Notch and binding of natural ligand is partly and preferably completely blocked/inhibited and as a consequence Notch pathway activation (is at least in part) inhibited. Examples of dominant negative ligand are for example mutants of Delta and Serrate comprising intracellular deletions (Sun and Artavanis-Tsakonas, 1996).

In another preferred embodiment said ligand mediated activation of Notch is at least in part diminished by providing said animal with a dominant negative Notch. In principle each type of Notch molecule may be used for this purpose, i.e. Notch1, 2, 3 or 4 or a functional fragment and/or a functional derivative thereof. A functional fragment is any fragment (N-terminal fragment, C-terminal fragment or an internal fragment or any combination thereof) derived from either of these molecules (or equivalent thereof) which is capable of binding to Notch ligand. Such a functional fragment may for example be present as a membrane or as a non-membrane bound compound. By binding of the dominant negative Notch to the available ligands, said ligand cannot bind to naturally/originally/functionally available Notch and hence Notch pathway activation is at least in part inhibited. A functional derivative is for example a Notch molecule which has been mutated (point mutation, insertions) such that binding to a ligand is still possible but that the mutation prevents the signal of ligand binding to be transmitted. A functional derivative may also be derived from another species. In yet another preferred embodiment said ligand mediated activation of Notch is at least in part diminished by providing said animal with an antibody capable of at least in part blocking the interacting between a Notch ligand and Notch. Such an antibody is for example directed to the ligand binding part of Notch or directed to the part of the ligand that interacts with Notch. The production of antibodies is routine within in the art and hence no further details regarding this matter is provided. Independent on the type of used inhibition of ligand mediated activation of Notch, the result is the same: the formation of (at least part of) NICD is inhibited, which eventually results in the formation of postmitotic cells (for example goblet cells) from transformed/malignant cells.

As already indicated, inhibition (of at least part of, but preferably completely) of Notch pathway activation is accomplished in different ways. In yet another preferred embodiment, the invention provides a method for at least in part decreasing intestinal adenoma and/or adenocarcinoma present in an animal comprising at least in part inhibiting Notch pathway activation in said animal, wherein said Notch pathway activation is at least in part inhibited by providing said animal with an ADAM protease inhibitor. After binding of a Notch ligand to Notch, the Notch receptor undergoes a conformational change that allows ectodomain shedding through the action of an ADAM protease. ADAM stands for a disintegrin and metalloprotease. By providing a substance capable of inhibiting the protease that accomplishes the ectodomain shedding, the Notch pathway activation is at least partly but preferably completely inhibited, i.e. no formation of NICD occurs. This results, in the case of intestinal adenoma and/or adenocarcinoma, in a change from proliferative adenoma and/or adenocarcinoma cells into postmitotic, e.g. goblet cells. Again, local inhibition of Notch pathway activation is preferred to avoid, as much as possible, any possible undesired side effects.

It is clear to a person skilled in the art that also different ways of at least in part inhibiting Notch activation may be combined to, for example, increase efficacy.

Intestinal adenomas result from mutational activation of the Wnt pathway, most commonly due to the loss of the intestinal tumor suppressor gene APC (Kinzler and Vogelstein, 1996). We have recently determined the Wnt target gene program in colorectal cancer cells and thus unveiled a remarkable symmetry between adenoma cells and proliferative crypt progenitors (van de Wetering et al 2002). To investigate whether this symmetry extends to the Notch pathway, we studied the expression of various Notch pathway components and target genes in adenomas that spontaneously occur in the APC-mutant min mouse. In general, expression of receptors and ligands in adenomas closely followed the crypt expression as reported previously (Schroder and Gossler, 2002). As examples, FIG. 1 gives expression in adenomas of Notch2 and Delta-like-1 in adenomas of the APC-mutant min mouse. More importantly, HES1 expression, indicative of active Notch signalling, occurred not only in crypts, but was also observed uniformly in adenomas of all sizes in the intestines of APC^{min} mice (FIG. 1). This observation implied that, like in crypts, both the Notch and Wnt pathways were active in proliferative adenoma cells. The herein disclosed examples show unexpectedly that stem/progenitor cells of the intestinal epithelial need both Wnt and Notch signalling to be able to remain undifferentiated. It is herein disclosed that the same is true for transformed/malignant cells in which the Wnt cascade is constitutively active: Notch activity is needed to maintain the transformed/malignant state of a cell.

In a preferred embodiment the invention provides a method for at least in part decreasing intestinal adenoma and/or adenocarcinoma present in an animal comprising at least in part inhibiting Notch pathway activation in said animal, further comprising at least in part inhibiting Wnt pathway activation in said animal.

By providing an inhibitor of Wnt pathway activation to a cell suffering from an intestinal adenoma and/or adenocarcinoma, the malignant/non-transformed cell differentiates and dies shortly after its formation, hence decreasing the size/volume of an intestinal adenoma and/or adenocarcinoma. By a combined action of an inhibitor of Notch pathway activation as well as an inhibitor of Wnt pathway activation, cells of the intestinal adenoma and/or adenocarcinoma differentiate, resulting in at least partly diminishing of the intestinal malignancy. It is clear to a person skilled in the art that intestinal adenoma and/or adenocarcinoma may also be treated by the sole action of a Wnt pathway inhibitor.

In another embodiment the invention provides use of a Notch pathway inhibitor in the preparation of a medicament for the treatment of intestinal adenoma and/or adenocarcinoma. As already outlined above for the methods according to the invention, multiple inhibitors may be used to at least in part decrease the formation of NICD and hence at least in part prevent Notch pathway activation. Examples of preferred Notch inhibitors are: γ-secretase inhibitors, such as DAPT or dibenzazepine (DBZ) or benzodiazepine (BZ), an inhibitor capable of diminishing ligand mediated activation of Notch (for example via a dominant negative ligand of Notch or via a dominant negative Notch or via an antibody capable of at least in part blocking the interacting between a Notch ligand and Notch), or an inhibitor of ADAM proteases. Moreover, such an inhibitor may be supplemented with a Wnt pathway inhibitor or combined with already available therapy such as chemotherapy, surgery or irradiation.

In a preferred embodiment the invention provides use of a Notch pathway inhibitor in the preparation of a medicament for the treatment of intestinal adenoma and/or adenocarcinoma, wherein said intestinal adenoma and/or adenocarcinoma occur in patients with the hereditary syndrome familial adenomatous polyposis (FAP). FAP is caused by an inherited mutation in the adenomatous polyposis coli (APC) gene. Polyposis essentially means “many polyps”. Polyps are small growths of tissue on the wall of the intestine. The most common polyp and the only one that can become an adenocarcinoma is the adenomatous polyp. In FAP, hundreds to thousands of polyps develop throughout the colon. These polyps usually begin to form around the age of 16. Unless removed, these polyps almost always develop into cancer. Individuals diagnosed with an APC mutation are not only at risk for colorectal cancer but may also have an increased risk over the general population for other types of cancer. Attenuated FAP (AFAP) is a less severe variation of FAP in which a person develops less polyps. AFAP does not usually develop as early as FAP, but it carries a similarly high risk of cancer. Once FAP is diagnosed the patient is in the end typically subjected to a prophylactic (sub)total colectomy. Such a colectomy may now partly be skipped if a patient suffering from FAP is subjected to a method or use according to the invention. The herein used APC^{min} mice are in principle the counterpart of human FAP.

In yet another embodiment the invention provides a pharmaceutical composition comprising at least two Notch pathway activation inhibitors or comprising at least one Notch pathway activation inhibitor and at least one Wnt pathway inhibitor.

Pharmaceutical compositions according to the invention may be provided in the form of a powder, a solution or suspension in a(n) (non) aqueous liquid or as an emulsion. Said pharmaceutical may further comprise pharmaceutically acceptable carriers and/or diluents. The pharmaceutical compositions may be administered orally, parenterally, intramuscularly, intravenously, parentally, intraperitonally or colorectally (for example with a suppository). Oral and colorectal delivery is preferred because the to be treated areas are easily reached via these routes of delivery. Moreover, the delivery is preferably locally to avoid any undesired side effects.

With respect to the doses it is noted that a skilled person is capable, based on for example the already known pharmacokinetics, to determine an effective dosage of for example DAPT and/or DBZ and/or BZ. Moreover, by using one or multiple well-known dose-finding experiments, effective doses can also be determined by the skilled person.

Besides the use of a pharmaceutical in any of the outlined methods, a pharmaceutical according to the invention is also very useful as an additive (residual) to other therapies. For example, if it is decided to remove an adenoma or an adenocarcinoma with surgery, a pharmaceutical according to the invention is provided before and/or during and/or after surgery to induce the formation of a postmitotic cell from a adenoma and/or adenocarcinoma or to at least in part further decrease an adenoa and/or adenocarcinoma or to treat remaining (possibly non-visible) residual adenoma and/or adenocarcinoma cells. This is for example performed by rinsing the abdominal cavity with a fluidized pharmaceutical according to the invention or by directly injecting a pharmaceutical according to the invention in suspected areas.

The invention will be explained in more detail in the following description, which is not limiting the invention.

### EXPERIMENTAL PART

### Experiment I

Material and Methods

Antibodies for Immunohistochemistry.

The following antibodies were used. Mouse anti-Ki67 (1:100; Novocastra), mouse anti-β-catenin (1:50; Transduction Labs).

Tissue Sample Preparation, Immunohistochemistry and In Situ Hybridisation.

The intestinal tract was dissected as a whole and flushed gently with cold PBS to remove any faecal content followed by a flush with Formalin. The small intestine was rolled up into a compact circle and fixed in Formalin at RT for 16 hours. The tissues were sectioned (2-6 μm). Following dewaxing and hydration, sections were pretreated with peroxidase blocking buffer (120 mM Na₂HPO₄, 43 mM citric acid, 30 mM NaN₃, 0.2% H₂O₂; pH 5.8) for 15 minutes at room temperature. Antigen retrieval was performed by boiling samples in Na-citrate buffer (10 mM, pH 6.0). After 20 minutes, the boiling pan was allowed to slowly cool down to room temperature. Incubation of antibodies was performed in BSA in PBS overnight at 4° C. and at RT for 1 hour for Lysozyme, Ki67. In all cases, the Envision⁺ kit (DAKO) was used as a secondary reagent. The incubation time was 30 min. Stainings were developed using DAB. Slides were then counterstained with hematoxylin and mounted. The probes used for in situ hybridization are as described (Schroder et al 2002).

Similar in situ hybridisations are performed on well-characterised human pathological paraffin embedded samples, but obviously with human RNA sequences as probes.

Alcian Blue Staining

Following dewaxing and hydration, tissues were incubated for 5 min in Alcian Blue (1% in 0.5% acetic acid). Subsequently washed in water, 1 min incubated in Neutral Red. Quickly dehydrated, washed in Xylene and mounted with Pertex.

Pharmacological Inhibition of Notch Signaling in APC^{min} Mice

The 8-week-old APC^{min} mice were treated with two different, orally deliverable γ-secretase inhibitors (DAPT: 100 mg/kg in corn oil) for 2.5, 6.5 or 15 days, after which their intestines were isolated and examined histologically.

Results

Intestinal adenomas result from mutational activation of the Wnt pathway, most commonly due to the loss of the intestinal tumor suppressor gene APC (reviewed in Kinzler and Vogelstein, 1996; Bienz and Clevers, 2000). We have recently reported a remarkable symmetry between the colorectal cancer cells and proliferative crypt progenitors in terms of the expression of a Wnt target gene program (van de Wetering et al 2002). To investigate whether the symmetry between crypts and intestinal neoplasia extends to the Notch pathway, we studied the expression of various Notch pathway components and target genes in adenomas that spontaneously occur in the APC-mutant min mouse. In general, expression of receptors and ligands in adenomas closely followed the crypt expression as reported previously (Schroder and Gossler, 2002) (Table I). As examples, FIG. 1 shows expression of Notch2 and Delta-like-1 in adenomas. More importantly, Hes1 expression, indicative of active Notch signalling, occurred not only in crypts (Schroder and Gossler, 2002), but was also observed uniformly in adenomas of all sizes in the intestines of APC^{min} mice (FIG. 1, right panel). This observation implied that, like in crypts, the Notch and Wnt pathways are simultaneously active in proliferative adenoma cells.

We then asked whether Notch pathway activity was essential for the maintenance of the undifferentiated, proliferative phenotype of adenoma cells. We elected to inhibit the Notch pathway pharmacologically through the use of the γ-secretase inhibitor DAPT. We then initiated treatment of 15 week-old Apc^{min} mice that at this age carry 30-60 macroscopically detectable adenomas or polyps in the small intestine and 1-3 adenomas in the colon. The mice were treated with for up to 15 days, after which their intestines were examined histologically. Based on a pilot experiment and published treatment regimens, the Apc^{min} mice were orally dosed once daily with DAPT at 100 mg/kg in maize oil. No consistent effects were seen on day 2.5 or day 6.5 of treatment. However, extensive changes had occurred within the adenomas at day 15 of treatment with the compounds. As demonstrated in FIGS. 2 and 3, numbers of proliferating, Ki67-positive cells dramatically decreased. In FIG. 2, colon adenomas are visualized by beta-catenin staining (left panels). Of note, proliferation in adjacent, normal crypts was unaffected. Large foci of Alcian Blue-positive goblet cells had appeared throughout the adenomas. Nuclear β-catenin-positivity of the arrested and differentiated cells indicated that these derived from the Apc-negative adenoma. In FIG. 3 examples are given of adenomas in small intestine. The adenoma tissue again stains dark-brown in the left panels. Cell proliferation as evidenced by Ki67-staining (right panels), has halted upon treatment with the compound DAPT.

A wealth of evidence has implied the Wnt cascade as the major driving force behind the proliferative potential of transit amplifying crypt progenitor cells as well as of adenomas and adenocarcinomas of the intestine in mouse and man. The current data indicate that active Notch signalling plays an equally important role in the maintenance of the undifferentiated state of crypt progenitors and of Apc-mutant neoplastic cells. While the Wnt cascade is mutationally activated at the onset of the transformation process, the Notch pathway most likely remains non-mutated during tumor progression. It can be envisioned, though, that a strong selective pressure exists in advanced tumors for the presence of activating Notch pathway mutations. Such activating mutations have not been reported in colorectal cancer.

The Wnt cascade, as activated in colorectal cancer, is generally considered to present a rather unfavorable target for drug development, as the segment of the cascade downstream of the Apc tumor suppressor protein is driven entirely by protein-protein interactions. As directly demonstrated here, the Notch pathway represents an alternative targeted-drug strategy for the treatment of intestinal neoplasia such as Familial Adenomatous Polyposis or sporadic colorectal cancer. Multiple γ-secretase inhibitors of diverse chemical origins have been developed for the treatment of Alzheimer's disease. Increases in intestinal goblet cell numbers in animal toxicity studies have been noted as the principal unwanted side-effect of these compounds. Yet, we state that this Notch-related effect makes these γ-secretase inhibitors into attractive therapeutic modalities for colorectal cancer.

### Experiment II

Material and Methods

Antibodies for Immunohistochemistry

The following antibodies were used. Mouse anti-1167 (1:100; Novocastra), mouse anti-β-catenin (1:50; Transduction Labs), Rabbit anti-Math1 (1:50; a kind gift of Dr Jane Johnson).

Tissue Sample Preparation, Immunohistochemistry and In Situ Hybridization.

The intestinal tract was dissected as a whole and flushed gently with cold PBS to remove any fecal content followed by a flush with formalin. The small intestine was rolled up into a compact circle and fixed in Formalin at RT for 16 hours. The tissues were sectioned (2-6 mm). Following dewaxing and hydration, sections were pretreated with peroxidase blocking buffer (120 mM Na₂HPO₄, 43 mM citric acid, 30 mM NaN₃, 0.2% H₂O₂; pH 5.8) for 15 minutes at room temperature. Antigen retrieval was performed by boiling samples in Na-citrate buffer (10 mM, pH 6.0). After 20 minutes, the boiling pan was allowed to slowly cool down to room temperature. Incubation of antibodies was performed in BSA in PBS overnight at 4° C. for antibodies directed against Math1 and at RT for 1 hour for antibodies directed against Ki67 and β-catenin. In all cases, the Envision+ kit (DAKO) was used as a secondary reagent. Stainings were developed using DAB. Slides were counterstained with hematoxylin and mounted.

Gamma Secretase Inhibitor DBZ

Three grams of DBZ (Milano et al 2004) was custom-synthesized by Syncom, Groningen, the Netherlands to >99.9% purity. DBZ was suspended finely in 0.5% (w/v) Hydroxypropyl Methylcellulose (Methocel E4M) and 0.1% (w/v) Tween 80 in water

Treatment of Animals with the Gamma Secretase Inhibitor DBZ.

C57B16 mice were injected i.p. with the gamma secretase inhibitor DBZ injected daily with 0, 3, 10, and 30 μmol/Kg for 5 days.

We initiated treatment of 8 week-old Apc^{min} mice, two mice each were treated with 0, 3, 10 or 30 μmol/Kg DBZ injections. DBZ was injected every 48 hours intraperitoneally for 10 days, after which intestines were examined histologically by serial sectioning.

Results

As an alternative tool to block Notch signalling in vivo, we synthesized the γ-secretase inhibitor DBZ 18 to >99.9% purity. DBZ blocked Notch-cleavage in a cell-based assay with an IC50 of <2 nM (not shown). In a dose-finding experiment, the compound was injected every day i.p. at 0, 3, 10, and 30 μmol/Kg in C57B16 mice for 5 days. At higher doses (10 and 30 μmol/Kg), Goblet cell conversion was complete after 5 days of i.p. injections as shown by PAS staining (FIGS. 4C and D, resp). Moreover, cell proliferation had entirely halted and histological markers (Ki67 and Math1) revealed that the tissue changes were indistinguishable from those observed upon CSL deletion (results not shown). At 3 μmol/Kg goblet cell numbers slightly increased as shown by PAS staining. (FIG. 4B). The combined genetic and pharmacological findings show that Notch signalling is essential within the crypt compartment proper to maintain the undifferentiated state of the crypt progenitors.

In questioning whether Notch pathway activity was essential for the maintenance of the undifferentiated, proliferative phenotype of adenoma cells, we elected to inhibit the Notch pathway pharmacologically. In experiment I this was effected through the use of the γ-secretase inhibitor DAPT. In experiment II we elected to inhibit the Notch pathway pharmacologically through the use of the γ-secretase inhibitor DBZ (Wong et al 2004). We initiated treatment of 8 week-old Apc^{min} mice, which at this age carry 30-60 macroscopically detectable adenomas (polyps) in the small intestine and 0-3 adenomas in the colon. Two mice each were treated with 0, 3, 10 or 30 μmol/Kg DBZ for 10 days, after which intestines were examined histologically by serial sectioning. Staining for β-catenin delineated the adenomas, which were often embedded in an accumulation of hyperplastic yet untransformed normal crypts (FIGS. 5A, C). DBZ at 10 or 30 μmol/Kg readily induced Math1+/PAS+/Ki67-Goblet cells within adenomas (FIGS. 5D, M-O), while the effects at 3 μmol/Kg were minimal, as were the effects on normal crypts (not shown). Different conversion rates were observed in individual adenomas, even within the same animal. To quantify the conversion rate, 100 adenomas from mice treated with 10 μmol/Kg DBZ were analyzed by determining the percentage of Math1+ nuclei. In 8% of the adenomas, >50% of all epithelial cells converted into Math1+ cells. In 20% of the adenomas 10-50% conversion occurred; 28% showed 1-10% conversion, while 46% showed no Goblet cell conversion. Goblet cell conversion was never observed in untreated Apc^{min} mice: In each of 100 adenomas analyzed, <1% Math1+Goblet cells were observed. The observations show that adenoma cells can be forced to differentiate upon Notch pathway inhibition.

### Experiment III

Material and Methods

Antibodies for Immunohistochemistry

The following antibodies were used. Mouse anti-Ki67 (1:100; Novocastra), mouse anti-βcatenin (1:50; Transduction Labs).

Tissue Sample Preparation and Immunohistochemistry.

The intestinal tract was dissected as a whole and flushed gently with cold PBS to remove any fecal content followed by a flush with formalin. The small intestine was rolled up into a compact circle and fixed in Formalin at RT for 16 hours. The tissues were sectioned (2-6 mm). Following dewaxing and hydration, sections were pretreated with peroxidase blocking buffer (120 mM Na₂HPO₄, 43 mM citric acid, 30 mM NaN₃, 0.2% H₂O₂; pH 5.8) for 15 minutes at room temperature. Antigen retrieval was performed by boiling samples in Na-citrate buffer (10 mM, pH 6.0). After 20 minutes, the boiling pan was allowed to slowly cool down to room temperature. Incubation of antibodies was performed in BSA in PBS overnight at RT for 1 hour for antibodies directed against Ki67 and β-catenin. In all cases, the Envision+ kit (DAKO) was used as a secondary reagent. Stainings were developed using DAB. Slides were counterstained with hematoxylin and mounted.

Gamma Secretase Inhibitor DBZ and BZ

Three grams of DBZ and BZ (Milano et al 2004) was custom-synthesized by Syncom, Groningen, the Netherlands to >99.9% purity. DBZ was suspended finely in 0.5% (w/v) Hydroxypropyl Methylcellulose (Methocel E4M) and 0.1% (w/v) Tween 80 in water and BZ was suspended finely in 6% (v/v) Ethanol/94% (v/v) Labrafil M 1944 CS.

Treatment of Animals with the Gamma Secretase Inhibitor DBZ and BZ.

C57B16 mice were injected i.p. with the gamma secretase inhibitor BZ injected daily with 0, 3, 10 and 30 μmol/Kg for 5 days.

For the oral study, the drugs (DBZ and BZ) were administered to 8 week-old Apc^{min} mice (10, 20 or 30 μmol/Kg) after which intestines were examined histologically by serial sectioning.

Results

As an alternative tool to block Notch signalling in vivo, we also synthesized the γ-secretase inhibitor BZ to >99.9% purity. BZ and DBZ both blocked Notch-cleavage in a cell-based assay with an IC50 of 2.2 and 1.7 nM, resp. (not shown). In a dose-finding experiment, the BZ compound was injected every day i.p. at 0, 3, 10, and 30 μmol/Kg in C57B16 mice for 5 days. At higher doses (10 and 30 μmol/Kg), Goblet cell conversion was complete after 5 days of i.p. injections as shown by PAS staining. The tissue changes were indistinguishable from those observed upon CSL deletion (results not shown) and after treatment with DBZ (see experiment II). These data again confirmed that Notch signalling is essential within the crypt compartment proper to maintain the undifferentiated state of the crypt progenitors.

In questioning whether Notch pathway activity was essential for the maintenance of the undifferentiated, proliferative phenotype of adenoma cells, we elected to inhibit the Notch pathway pharmacologically. In experiment I this was effected through the use of the γ-secretase inhibitor DAPT. In experiment II we elected to inhibit the Notch pathway pharmacologically through the use of the γ-secretase inhibitor DBZ after IP administration. In experiment III we elected to inhibit the Notch pathway pharmacologically through the use of different concentrations of the γ-secretase inhibitor DBZ and BZ after oral administration. The rational behind the usage of different concentration is that high concentration of these compounds may not only affect the intestinal tumor, but also the normal tissue.

We initiated oral treatment of 8 week-old Apc^{min} mice, which at this age carry 30-60 macroscopically detectable adenomas (polyps) in the small intestine and 0-3 adenomas in the colon. Three mice each were orally treated with 10, 15 or 30 μmol/Kg BZ or DBZ for max. 12 days, after which intestines were examined histologically by serial sectioning. Staining for β-catenin delineated the adenomas, which were often embedded in an accumulation of hyperplastic yet untransformed normal crypts. Changes had occurred within the adenomas at day 12 of oral treatment with the compounds BZ and DBZ. At a concentration of 15 μmol/Kg the number of proliferating, Ki67-positive cells in the adenomas were dramatically decreased, while the majority of normal intestinal tissue seems to be unaffected.

In conclusion, the cell proliferation in adenomas after oral treatment of APC^{min} mice with the compounds BZ and DBZ, as evidenced by Ki67/β-catenin staining, has halted while normal tissue seems to be unaffected.

TABLE I

| gene | Crypt epithelium | Adenomas |
|---|---|---|
| Notch1 | +/− | +/− |
| Notch2 | + | + |
| Notch3 | + | + |
| Notch4 | − | +/− |
| Delta1 | ++ | + |
| Delta3 | + | + |
| Delta4 | + | + |
| Jagged1 | +/− | +/− |
| Jagged2 | − | − |
| Hes1 | + | + |

### DESCRIPTION OF FIGURES

FIG. 1

Expression of examples of Notch pathway components (Delta-like 1 (left panel)), Notch2 (middle panel), and a target gene of the activated Notch pathway (Hes1 (right panel)) in adenomas of APC^{min} mice, as demonstrated by in situ hybridisation.

FIG. 2

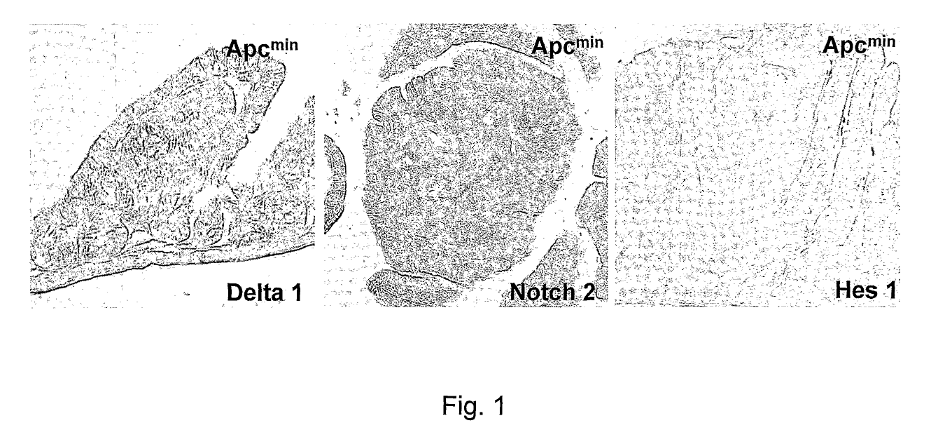

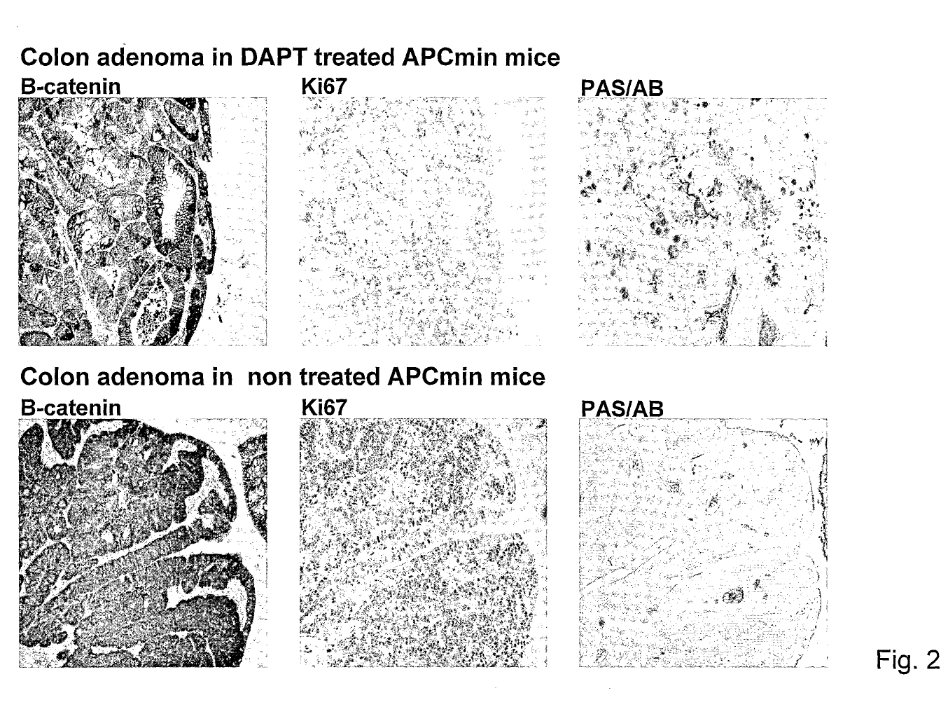

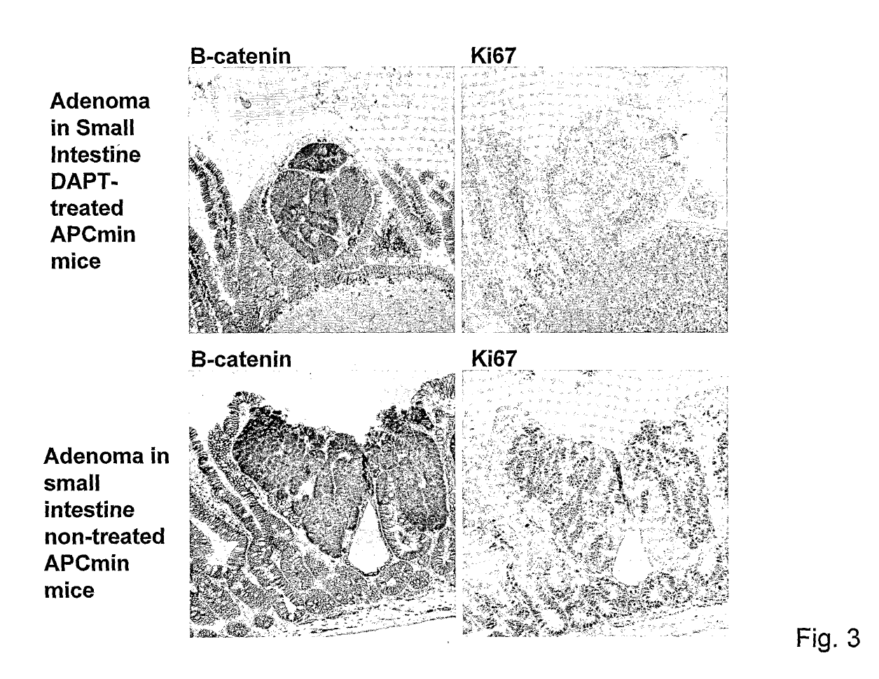

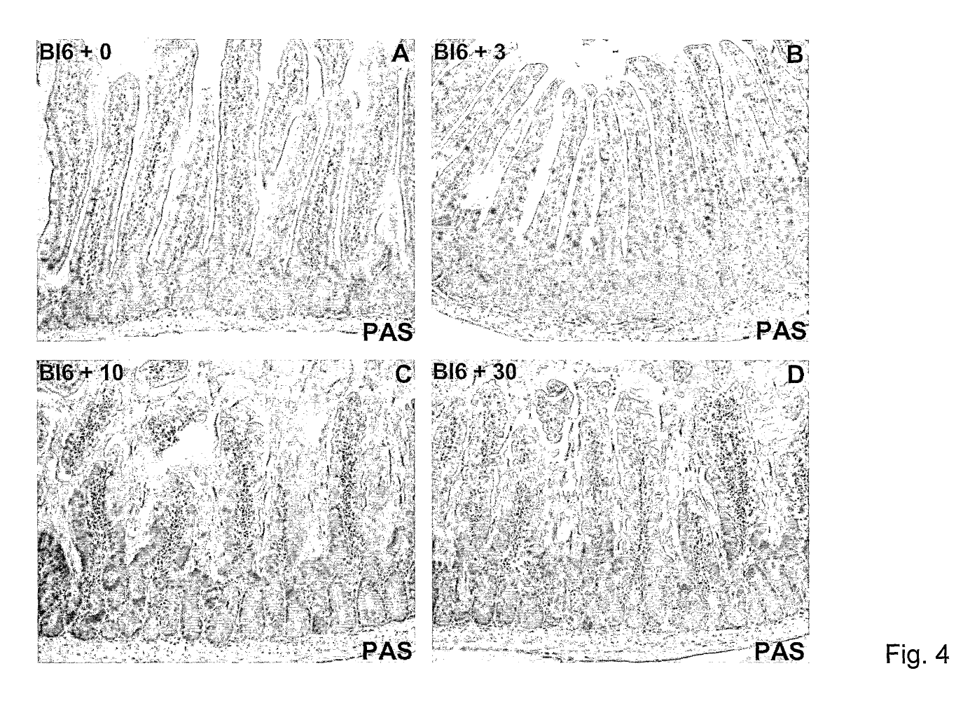

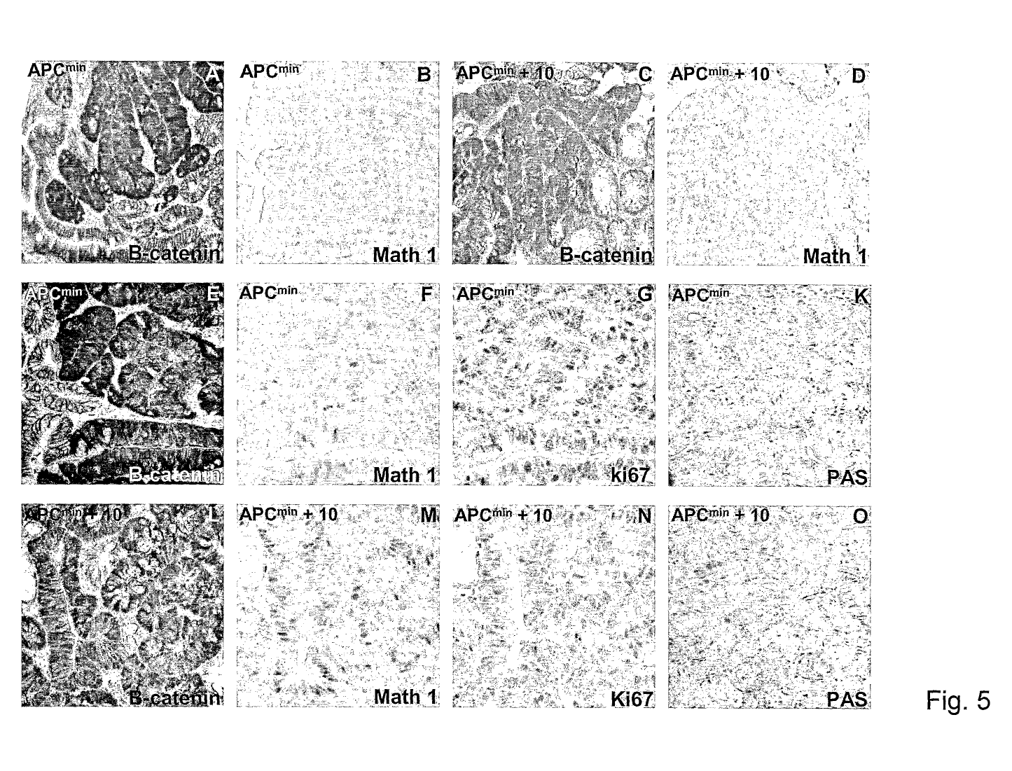

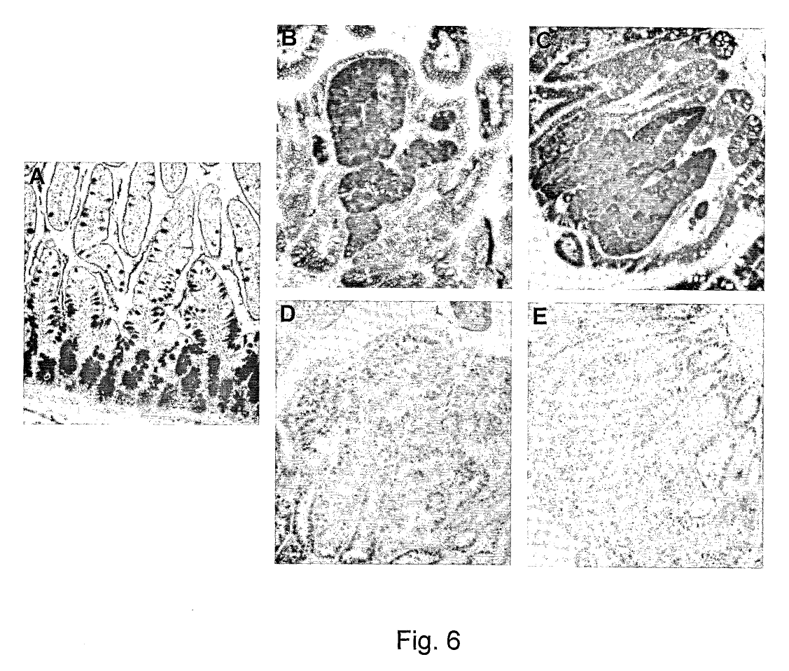

Analysis of colon adenomas of APC^{min} mice treated (top panels) or not treated (bottom panel) with DAPT as indicated in the text. Dark staining in the left top/bottom panels reveals regions in the epithelium with high levels of beta-catenin, representing the adenoma tissue. Middle top/bottom panels give staining for the cell cycle/proliferation marker Ki67. Note that treatment with DAPT leads to strongly diminished proliferation activity in the adenoma. Right top/bottom panels show the increase in goblet cells upon DAPT treatment as indicated by the dark-staining cells upon Alcian blue stain.

FIG. 3

Analysis of small intestinal adenomas of APC^{min} mice treated (top panels) or not treated (bottom panel) with DAPT as indicated in the text. Dark staining in the left top/bottom panels reveals regions in the epithelium with high levels of beta-catenin, representing the adenoma tissue. Right top/bottom panels give staining for the cell cycle/proliferation marker Ki67. Note that treatment with DAPT leads to strongly diminished proliferation activity in the adenoma.

FIG. 4

Conversion of proliferative crypt cells into post-mitotic goblet cells by the gamma secretase inhibitor DBZ.

C57B16 mice were injected i.p. with the gamma secretase inhibitor DBZ injected daily with 0, 3, 10, and 30 μmol/Kg for 5 days. At 3 μmol/Kg Goblet cell numbers slightly increased as shown by PAS staining (B), while with 10 and 30 μmol/Kg, the conversion of proliferative crypt cells into post-mitotic Goblet cell was complete (C and D, resp.).

FIG. 5

Conversion of proliferative cells in the APCmin tumor into post-mitotic goblet cells by the gamma secretase inhibitor DBZ.

APCmin mice were treated with 0 μmol/Kg DBZ (A,B,E,F,G,K) or 10 μmol/Kg DBZ (C,D,L,M,N,O) for 10 days, after which intestines were examined histologically by serial sectioning. Staining for β-catenin delineated the adenomas (Fig. A,C,E,L). DBZ treatment induced Math1 (D versus B and, at an higher magnification, M versus F) and Pas expression (O versus K), and reduced Ki67 expression (N versus G).

FIG. 6

Analysis of the intestine and intestinal adenomas of APC^{min} mice treated or not treated with BZ as indicated in the text.

C57B16 mice were injected i.p. with the gamma secretase inhibitor BZ injected daily with 0, 3, 10, and 30 μmol/Kg for 5 days. At 3 μmol/Kg Goblet cell numbers slightly increased as shown by PAS staining (not shown), while with 10 and 30 μmol/Kg, the conversion of proliferative crypt cells into post-mitotic Goblet cell was complete (A).

Analysis of adenomas of APC^{min} mice treated (top panels) or not treated (bottom panel) by oral administration with BZ or DBZ as indicated in the text. Dark staining in panels (B,C) reveals regions in the epithelium with high levels of beta-catenin, representing the adenoma tissue. Panels (D,E) give staining for the cell cycle/proliferation marker Ki67. Note that treatment with DBZ (not shown) or BZ (panel E versus D) leads to strongly diminished proliferation activity in the adenoma, while surrounding tissue seems to be unaffected

### REFERENCES

Artavanis-Tsakonas S, R and M D, Lake R J. Notch signalling: cell fate control and signal integration in development. Science. 1999 Apr. 30; 284(5415):770-6.
Baron M. An overview of the Notch signalling pathway. Semin Cell Dev Biol. 2003 April; 14(2):113-9.
Bienz M. and Clevers, H. (2000) Linking colorectal cancer to Wnt signaling. Cell. October 13; 103(2):311-320.
De Strooper B, Annaert W, Cupers P, Saftig P, Craessaerts K, Mumm J S, Schroeter E H, Schrijvers V, Wolfe M S, Ray W J, Goate A, Kopan R. Presenilin-1-dependent gamma-secretase-like protease mediates release of Notch intracellular domain. Nature. 1999 Apr. 8; 398(6727):518-22.
Harrison T. et al. (2004) γ-Secretase as a target for drug intervention in Alzheimer's disease, Current Opinion in Drug Discovery & Development, 7(5); 709-719.
Heitzler P, Bourouis M, Ruel L, Carteret C, Simpson P. Genes of the Enhancer of split and achaete-scute complexes are required for a regulatory loop between Notch and Delta during lateral signalling in Drosophila. Development. 1996 January; 122(1):161-71.
Jensen J, Pedersen E E, Galante P, Hald J, Heller R S, Ishibashi M, Kageyama R, Guillemot F, Serup P, Madsen O D. Control of endodermal endocrine development by Hes-1. Nat Genet. 2000 January; 24(1):36-44.
Kinzler, K. W. and Vogelstein B., (1996) Lessons from hereditary colorectal cancer. Cell, October 18; 87(2):159-170.
Kopan R, Goate A. A common enzyme connects notch signaling and Alzheimer's disease. Genes Dev. 2000 Nov. 15; 14(22):2799-806.
Milano J, McKay J, Dagenais C, Foster-Brown L, Pognan F, Gadient R, Jacobs R T, Zacco A, Greenberg B, Ciaccio P J. Modulation of Notch Processing by {gamma}-Secretase Inhibitors Causes Intestinal Goblet Cell Metaplasia and Induction of Genes Known to Specify Gut Secretory Lineage Differentiation. Toxicol. Sci. 2004 November; 82(1):341-358.
Mumm J S, Kopan R. Notch signalling: from the outside in. Dev Biol. 2000 Dec. 15; 228(2):151-65.
Oellers N, Dehio M, Knust E. bHLH proteins encoded by the Enhancer of split complex of Drosophila negatively interfere with transcriptional activation mediated by proneural genes. Mol Gen Genet. 1994 Sep. 1; 244(5):465-73.
Schroder N and Gossler A. Expression of Notch pathway components in fetal and adult mouse small intestine. Gene Expr Patterns. 2002 December; 2(3-4):247-50.
Searfoss, G. H. et al (2003) Adipsin, a biomarker of gatrointestinal toxicity mediated by a functional gamma-secretase inhibitor, J. Biol. Chem., November 14 (278(46):46107-46116.
Sun, X. and Artavanis-Tsakonas, S. (1996) The intracellular deletions of Delta and Serrate define dominant negative forms of the Drosophila Notch ligands, Development, 122, pages 2465-2474.
Van de Wetering M, Sancho E, Verweij C, de Lau W, Oving I, Hurlstone A, van der Horn K, Batlle E, Coudreuse D, Haramis A P, Tjon-Pon-Fong M, Moerer P, van den Born M, Soete G, Pals S, Eilers M, Medema R, Clevers H. The beta-catenin/TCF-4 complex imposes a crypt progenitor phenotype on colorectal cancer cells. Cell. 2002 Oct. 18; 111(2):241-50.
Wong G T, Manfra D, Poulet F M, Zhang Q, Josien H, Bara T, Engstrom L, Pinzon-Ortiz M, Fine J S, Lee H J, Zhang L, Higgins G A, Parker E M. Chronic treatment with the gamma-secretase inhibitor LY-411,575 inhibits beta-amyloid peptide production and alters lymphopoiesis and intestinal cell differentiation. J. Biol Chem 2004 Mar. 26; 279(13):12876-12882.
Yang Q, Bermingham N A, Finegold M J, Zoghbi H Y. Requirement of Math1 for secretory cell lineage commitment in the mouse intestine. Science. 2001 Dec. 7; 294(5549):2155-8.
Zheng J. L. et al. (2000) Hes1 is a negative regulator of inner ear hair cell differentiation. Development. November; 127(21):4551-1560.

1. A method for modifying the fate of an adenoma and/or adenocarcinoma cell, said method comprising:
influencing Notch pathway activation in the adenoma and/or adenocarcinoma cell.

2. The method according to claim 1, so as to induce formation of a postmitotic cell from the adenoma and/or adenocarcinoma cell by at least in part inhibiting the Notch pathway activation in said adenoma and/or adenocarcinoma cell.

3. A method for at least in part decreasing an intestinal adenoma and/or adenocarcinoma present in an animal, said method comprising:
at least in part inhibiting Notch pathway activation in said animal.

4. The method according to claim 1, wherein said Notch pathway activation is at least in part inhibited by at least in part diminishing ligand mediated activation of Notch.

5. The method according to claim 1, wherein said Notch pathway activation is at least in part inhibited by providing an inhibitor of γ-secretase.

6. The method according to claim 5, wherein said inhibitor of γ-secretase is DAPT or dibenzazepine (DBZ) or benzodiazepine (DB).

7. The method according to claim 4, wherein said ligand mediated activation of Notch is at least in part diminished by providing a dominant negative ligand of Notch.

8. The method according to claim 4, wherein said ligand mediated activation of Notch is at least in part diminished by providing a dominant negative Notch.

9. The method according to claim 4, wherein said ligand mediated activation of Notch is at least in part diminished by providing an antibody capable of at least in part blocking the interacting between a Notch ligand and Notch.

10. The method according to claim 1, wherein said Notch pathway activation is at least in part inhibited by providing an ADAM protease inhibitor.

11. The method according to claim 1, further comprising at least in part inhibiting Wnt pathway activation.

12. A method of treating a subject for an intestinal adenoma and/or adenocarcinoma, said method comprising:
diagnosing the subject to determine whether the subject is suffering from or is at risk for suffering from an intestinal adenoma and/or adenocarcinoma; and
inhibiting such a subject's Notch pathway so as to inhibit intestinal adenoma and/or adenocarcinoma.

13. The method according to claim 12, wherein a γ-secretase inhibitor is administered to the subject.

14. The method according to claim 14, wherein said γ-secretase inhibitor is selected from the group consisting of DAPT, dibenzazepine (DBZ) and a benzodiazepine (DB).

15. The method according to claim 13, wherein said γ-secretase inhibitor diminishes ligand mediated activation of Notch in the subject.

16. The method according to claim 15, wherein said γ-secretase inhibitor is a dominant negative ligand of Notch.

17. The method according to claim 15, wherein said γ-secretase inhibitor is a dominant negative Notch.

18. The method according to claim 13, wherein said γ-secretase inhibitor at least partially blocks an interaction between a Notch ligand and Notch.

19. The method according to claim 13, wherein said γ-secretase inhibitor is an ADAM protease inhibitor.

20. The method according to claim 13, further comprising a Wnt pathway inhibitor.

21. The method according to claim 13, wherein the subject has been diagnosed with hereditary syndrome familial adenomatous polyposis (FAP).

22. A pharmaceutical composition comprising:
a Notch pathway activation inhibitor, and
another Notch pathway activation inhibitor or an Wnt pathway inhibitor.
